(19) United States
(12) Patent Application Publication
(10) Pub. No.: US 2009/0192542 A1
(43) Pub. Date: Jul. 30, 2009

(54) SINGLE BALLOON RIPENING DEVICE WITH NOVEL INSERTER AND INFLATOR

(75) Inventor: Steven B. Harter, Las Vegas, NV (US)

Correspondence Address: LAW OFFICES OF PAUL E. KUDIRKA, 40 BROAD STREET, SUITE 300, BOSTON, MA, 02109, US

(73) Assignee: APPLE MEDICAL CORPORATION, Marlboro, MA (US)

(21) Appl. No.: 12/417,063

(22) Filed: Apr. 2, 2009

Related U.S. Application Data
(63) Continuation of application No. 11/237,014, filed on Sep. 28, 2005.
(60) Provisional application No. 60/671,323, filed on Apr. 14, 2005.

(51) Int. Cl. A61M 25/10

(52) U.S. Cl. 606/193

(57) ABSTRACT

An improvement on the extra-amniotic balloon and technique of placement is disclosed. It involves a large capacity balloon, modified to efficiently place pressure on the cervix, and markers for ease and of placement. The malleable introducing stylet allows for safe and easy insertion, either manually or visually, through a cervix that is 0.5 cm or more in dilation. The optional traction collar can aid in more efficiently placing pressure on the cervix. The automatic pump for instillation of the fluid has a forward and reverse mode to allow for deflation and repositioning of the balloon if placement is less than optimal. This process can be performed by a physician or a nurse, already certified in the placement of fetal scalp electrodes or intrauterine pressure catheters. Additionally, the procedure can be performed by one person.

### CROSS-REFERENCE TO RELATED APPLICATIONS

This application claims priority of U.S. provisional patent application Ser. No. 60/671,323, filed Apr. 14, 2005.

### FEDERALLY SPONSORED RESEARCH OR DEVELOPMENT

Not Applicable.

### TECHNICAL FIELD

This invention relates to a single balloon ripening device with a novel inserter and inflator.

### BACKGROUND OF THE INVENTION

It has been noted that the incidence of labor inductions over the course of the last decade or more, has increased dramatically. Studies have shown that approximately one half of all inductions are accompanied by an unfavorable cervix, thus making it necessary for the increase use in cervical ripening methods. Labor induction is the process of stimulating uterine contractions before they occur naturally. This may either be out of necessity or convenience. Many working mothers want to know when they will deliver, so that they can make arrangements for time off, child care, and scheduling home support. Some conditions placing the fetus at risk (i.e. pregnancy induced hypertension, intrauterine growth restriction, and suspicious antenatal testing) also require early or timely delivery. Delivery at small community hospitals, which don't have full staffing after hours, is best done during the day. It has been declared that the optimal cervical-ripening agent has not yet been found.

An aid to determining the success of a labor induction is the Bishop's Score. The Bishop's Score has a scoring system based on initial dilation, effacement, fetal station, and cervical consistency and position. In order to have a successful labor induction, it is important that one has a Bishop's score of greater than 5 (scored for cervical dilation, effacement, station, consistency, and position). If the total score is more than 8, the probability of vaginal delivery after labor induction is similar to that after spontaneous labor. In order for a labor induction to be successful, the cervix, if unripe, must be ripened.

Currently, the most common method of cervical ripening is by means of prostaglandins, which can be taken orally (misoprostol) versus intracervical (dinoprostone). The problem with these methods is that they need to be utilized in the hospital, thus tying up labor facilities and nursing resources. The dosing schedule can either be once (Cervidil) or every 4-6 hours. The popularity of the Cervidil dinoprostone insert is in part due to the fact that many nurses are capable for placement, and with a telephone call, the practitioner can instruct the nurse to insert the device, and then start labor after the requisite 12 hours have passed. Since the prostaglandin methods can cause uterine hyper stimulation, they require continuous monitoring for at least 2-6 hours. This requires hospital admission, placement of IV access, and restriction of the mother's mobility and diet. These methods must be used with caution in women with prior uterine surgery, since there is a greater risk of uterine rupture.

It has been shown that mechanical dilation methods are just as effective as prostaglandins in ripening the cervix. These include hygroscopic dilators, osmotic dilators (Laminaria japonicum) and the cervical balloon devices (24-French Foley balloon, and the Atad Ripener Device). Laminaria, however, have been found to increase the risk of infection around the time of delivery, when compared to the prostaglandin and the balloon methods. Some studies have shown the balloon method to be more effective than the prostaglandin one, and that the cesarean section rate is less, especially in the nulliparous patient.

The practice of extra-amniotic balloon placement is not widespread, largely due to the difficulty involved in placement of the device. Many practitioners have never been trained in its use. Standard Foley catheters are commonly used, however, the length of the catheter tips and the difficulty of placing a pliable catheter though the cervix have been limiting factors. Trans cervical variations of the balloon design have been made (Atad, Cowan); however, the process of insertion has not been made any easier or more convenient. Supracervical placement of the balloon device has been shown to be effective, however modifications of the balloon can make it more efficient. Typically, balloon placement needs to be done by a physician under a sterile set-up, and often it requires the assistance of a nurse to hand instruments and to help inflate the device. The nature of the pregnant vagina to prolapse inward and occlude the view of the cervix, and the problem with other factors such as patient obesity, and prior surgical scarring of the cervix, can make this a daunting task.

Additionally, the inflation and deflation of the balloon can be difficult, especially if a larger capacity syringe is used. It almost always requires the presence of an assistant for inflation.

Other background information can be found in the following references:
Rayburn, William F., MD, “Sorting Through New Data on Cervical Ripening”, OBG Management, November 2001.
Atad, Jack, MD, et al. “Instruments & Methods”, Obstetrics & Gynecology, vol. 77, No. 1, January 1991.
Trofatter, Kenneth F., Jr., PhD, MD. “Cervical Ripening”, Clinical Obstetrics and Gynecology, vol. 35, No. 3, September 1992.
Rouben, Divya, MD and Fernando Arias, MD, PhD. “A Randomized Trial of Extra-Amniotic Saline Infusion Plus Intracervical Foley Catheter Balloon Versus Prostaglandin E.sub.2 Vaginal Gel for Ripening the Cervix and Inducing Labor in Patients With Unfavorable Cervices”, Obstetrics & Gynecology, vol. 82, No. 2, August 1993.
James, C, et al. “Use of the Foley catheter as a cervical ripening agent prior to induction of labor”, International Journal of Gynecology & Obstetrics, 57 (1994) 299-232.
St. Onge, Rick D., MD and Gregory T. Connors, MD. “Preinduction cervical ripening: A comparison of intracervical prostaglandin E.sub.2 gel versus the Foley catheter”, American Journal of Obstetrics & Gynecology, vol. 172, No. 2, Part 1, 1995.
Atad, Jack, MD, et al. “A Randomized Comparison of Prostaglandin E.sub.2, Oxytocin, and the Double-Balloon Device in Inducing Labor”, Obstetrics & Gynecology, vol. 87, No. 2, February 1996.

### SUMMARY OF THE INVENTION

The invention provides a balloon catheter for insertion into the uterus, said catheter having a lumen, a proximal end and a distal end, and said catheter comprising (a) an inflatable balloon portion connected to the lumen and positioned close to the distal end, and (b) a marker portion having visual or tactile markings positioned proximal to the balloon portion. The balloon portion can be inflated by the flow of fluid through the lumen. The marker may be an integral part of the catheter or may be permanently or temporarily affixed to the exterior of the catheter. In exemplary embodiments, the marker may be a colored or raised band, or may be an area of different texture compared to the rest of the exterior of the catheter. The distal end of the catheter distal to the balloon portion is preferably less than 1 cm in length, for example between about 5 to 8 mm in length. The marker may be, for example, between about 2 to 3 cm proximal to the balloon portion.

The catheter may optionally further comprise an inflation port controlling the flow of fluid through the lumen into the balloon portion. The catheter may also optionally comprise a pressure gauge which determines the pressure inside the balloon. The catheter may also optionally comprise a second lumen that allows flow of fluid through the catheter to or from the uterus.

Also provided are kits comprising the balloon catheter of the invention, which may also include a semi-rigid, or rigid stylet. The stylet may be composed of plastic or plastic over a malleable or flexible metal core. The diameter of the stylet is less than the diameter of the catheter, allowing for ease of stylet removal. Preferably the distal end of the stylet is cushioned and devoid of metal in order to avoid trauma to fetal membranes or scalp. The kit may further comprise a traction collar for placement around the shaft of the balloon catheter, preferably at a position distal to the marker.

### BRIEF DESCRIPTION OF THE DRAWINGS

FIG. 1A shows a catheter, with a marker, and having a balloon in a deflated state. The inflation port is also demonstrated.

FIG. 1B shows the catheter of FIG. 1A with the balloon in an inflated state.

FIG. 1C shows an end view of the catheter of FIG. 1A.

FIG. 1D is a sectional view of the catheter of FIG. 1A, taken along line 1D-1D of FIG. 1A, illustrating the double lumen nature of the catheter.

FIG. 2 shows the catheter of FIG. 1A having a stylet with a malleable core, tab and cushion tip.

FIG. 3 shows a motorized pump with a disposable control syringe and tubing attached.

FIG. 4A shows an optional traction collar made of soft foam rubber or silicone with internal one-way tines attached to the shaft of the catheter.

FIG. 4B shows a recessed hinge and latch allowing opening of the collar for ease of attachment.

FIG. 4C shows the end-on view of the traction collar in a closed state

### DETAILED DESCRIPTION OF A PREFERRED EMBODIMENT

While this invention is susceptible of embodiments in many different forms, there is shown in the drawings and will herein be described in detail, a preferred embodiment of the invention with the understanding that the present disclosure is to be considered as an exemplification of the principles of the invention and is not intended to limit the broad aspects of the invention to the embodiment illustrated.

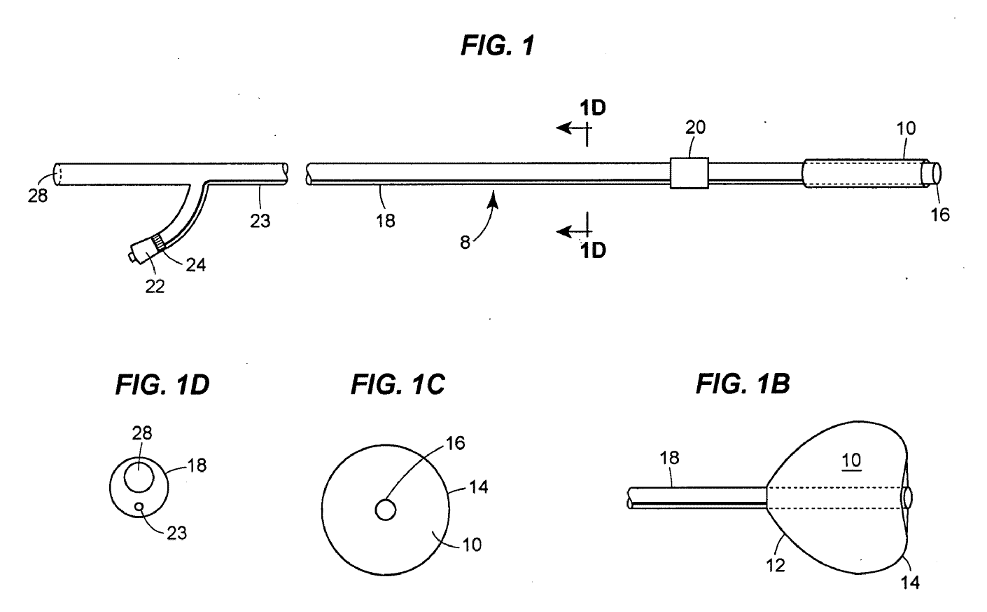

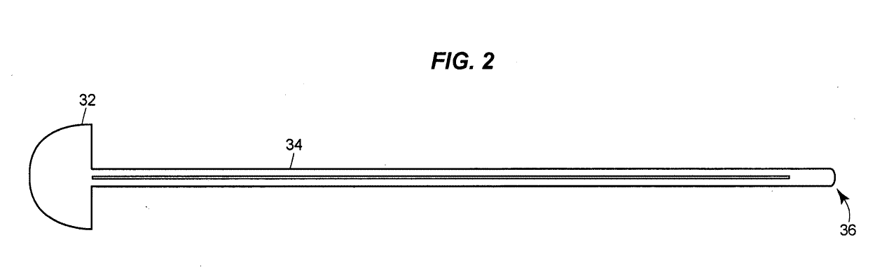

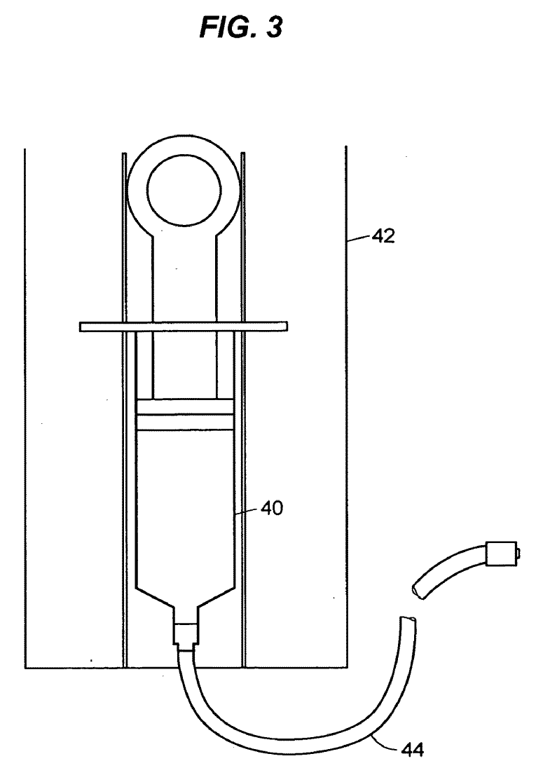

The present invention provides an improved method for insertion of a specialized single balloon catheter, generally designated 8, for cervical ripening. The catheter 8 itself may be 35-40 cm long and 16 to 24 french in diameter. FIG. 1A depicts an exemplary catheter 8 in the deflated state. As shown in FIG. 1B, the balloon 10 is tapered proximally 12, with a wider base distally 14, so that when inflated, it is in a heart or turnip shape. The approximate dimensions of the balloon when inflated are 5.5 to 6.0 cm in length and 13 to 15 cm in diameter at its widest end. The tapered end places more pressure on the cervix, while the wider end results in dissection of the amniotic membranes. The standard oblong or egg shape balloon can also be used. Variations on this design can be implemented. When the balloon is inflated, the proximal open tip 16 of the catheter 8 will be flush with, or slightly recessed from, the end of the balloon. Minimizing the length of the tip distal to the balloon aids in placement of the device. For example, it is easier to insert a catheter with a tip shorter than the typical 2-3 cm seen in a Foley catheter, preferably a tip less than 1 cm, or more preferably 5-8 mm.

The balloon 10 and inflation tube 18 are constructed of biocompatible materials that are elastic in nature, such as latex, or in the case of latex allergy, vinyl. The balloon inflates to a capacity able to accommodate 50-100 cc of fluid.

A stylet 30 inside the catheter 8 has a tab 32 at its proximal end to prevent insertion too far into the tube.

The catheter 8 will be fitted with a marker 20 that may take the form of a collar or colored or raised or textured band, which may be, for example, approximately 1.0 to 1.5 cm in length, so that tactically or visually, the depth of the distal end of the catheter 8 containing the balloon can be determined. The marker is preferably approximately 2-3 cm proximal to the end of the balloon 10 and allows the operator to know when the balloon is above the cervix and when it is safe to begin inflation.

An inflation port 22 provides fluid access to an inflation lumen 23 which extends to the balloon. The inflation port 22 may also be fitted with a simple pressure gauge 24 indicating when back pressure is too great, giving early indication of improper placement.

The catheter 8 also includes a second lumen 28 that extends the length of the catheter 8. The second lumen 28 receives a stylet 30. The second lumen 28 also allows for flow of fluid through to the uterus, so that spontaneous rupture of the fetal membranes can be immediately recognized. Additionally, the technique of hydro-dissection has been described as being potentially beneficial to speeding up the process of ripening, and the second lumen allows this to be accomplished. The stylet 30 inside the catheter 8 has a tab 32 at its proximal end to prevent insertion too far into the tube.

The stylet 30 will be comprised of a semi-rigid plastic, or plastic over a malleable, metal core 34, which can be molded into a position or shape that is most convenient for placement of the balloon through the cervix. The diameter of the stylet will be approximately 3 to 8 mm, depending on the diameter of the catheter 8, and an exemplary length is 40-45 cm. The distal end of the stylet 36 will be cushioned and devoid of metal, so as to avoid damage to a presenting head or to the fetal membranes.

A standard control syringe 40 or syringes with a collective 50-100 ml capacity is/are attached to the inflation port 22. For example, two 25 ml syringes may be utilized. The syringe may be operated manually or may be attached to a motorized pump unit 42, with IV tubing 44 extending from the end of the syringe to attach to the inflation port 22. The pump 42 is re-usable.

The control syringe 40 is filled with sterile saline or similar sterile liquid. The inflation port 22 includes an inflation valve which regulates fluid flow through the tube 18 to the proximal balloon 10. The motorized pump unit can be controlled remotely, either by hand or foot. The motorized pump unit can be operated in either direction to push fluid into the balloon or withdraw fluid from the balloon, to permit deflation, repositioning and re-inflation at a rate of, for example, 3 to 5 ml per second. Alternatively, a standard syringe could be used without a pump, but would require an assistant for inflation and deflation.

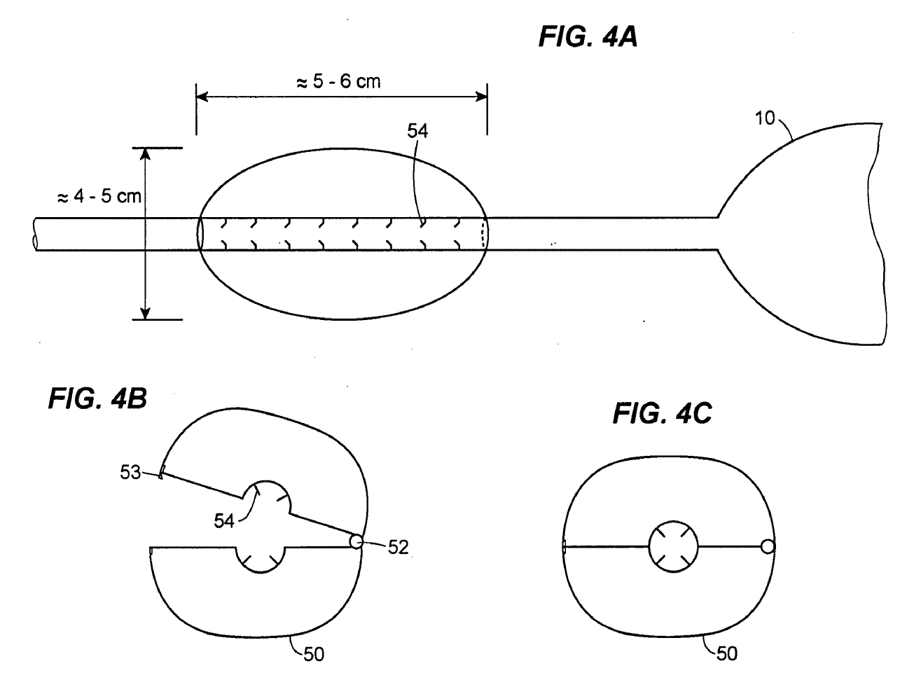

Referring to FIGS. 4A-4C, an egg-shaped traction collar 50 placed around the exterior of the catheter tubing in the vagina can also be utilized to place additional downward pressure on the cervix. The traction collar is preferably 5-6 cm in length and 4-5 cm in diameter. This traction collar 50 can be constructed of soft, pliable foam rubber. The traction collar 50 includes a recessed hinge 52 and a recessed latch 53, permitting the center of the traction collar 50 to open to one side, allowing it to slip on to the outside of the catheter tubing. The inner portion of the traction collar that contacts the catheter tubing will be fitted with one way blades 54, in order to allow the device to be moved forward easily along the catheter, but not to slip backwards. The traction collar 50 will be placed vaginally and can be left in the vaginal vault or slipped close to the cervix. The traction collar 50 will be no larger than the widest diameter of the balloon. The traction collar 50 is tapered in shape on both ends to allow for easy insertion and removal from the vagina.

The intracervical/extra-amniotic balloon catheter can be inserted prior to the induction of labor when an unripe cervix is present. The catheter is placed with the distal end containing the deflated balloon through the cervical canal and above the internal os. The band on the distal end of the catheter provides a tactile (for manual insertion) or visual (for placement with a vaginal retractor) marker, allowing for the operator to tell when the balloon has passed safely through the inner cervical os. The balloon catheter may then be inflated, paying close attention to patient comfort, with the automated pump or with an assistant using standard syringes.

Under sterile conditions, the stylet is loaded (or preloaded) inside the catheter tubing. The tubing from the automatic pump or standard syringe is attached to the inflation valve, and the balloon is tested for its integrity, as well as its ability to inflate and deflate easily. The patient is placed in dorso-lithotomy position and after coating the gloved hand with betadine (or other bacteriostatic solution), the cervix is checked, and a Bishop's score is assigned. With the examining hand still in place, the catheter can be threaded until the balloon end is through the cervix and the marker collar can be felt indicating the balloon tip is safely above the cervix. The catheter balloon is then slowly inflated. The patient will be informed that pressure sensation is normal, however indication of significant discomfort indicates that the balloon is in the cervical canal and not in the extra-amniotic space. Alternatively, if the inflation port is fitted with a simple pressure indicator, excessive backpressure can be monitored. Once completely inflated, the stylet can be removed, the inflation tubing unattached, and the catheter can be secured loosely to the patient's thigh or lower abdomen.

Alternatively, if desired, the patient can be placed in dorso-lithotomy position, and a sterile speculum can be used to visualize the cervix (after the digital cervical exam has been done). The cervix can be cleansed with bacteriostatic solution, and ring forceps can be placed at the level of the marker and the catheter can be inserted through the cervix to the proper depth. The forceps can be loosened, allowing for inflation of the balloon tip. If insertion is still difficult, an additional set of a ring forceps can be used to secure the cervix, allowing for greater ease of insertion, especially when dealing with a stenotic cervical os.

The extra-amniotic balloon has been successful when placed and labor induction is immediately started by administration of oxytocin, although a delay in time of 8 to 12 hours is preferred. The ripening process with balloon placement has, at times, been dramatic, causing marked dilatation within just a few hours. There have not been any studies as of yet, however, that have compared prior ripening to that of immediate induction of labor after placement of the device. The device can be placed in the office setting with minimal monitoring, and the patient can be free to pursue her normal activities.

What is claimed is:

1. A balloon catheter for insertion into a uterus, the catheter having a lumen, a proximal end and a distal end, and the catheter comprising (a) an inflatable balloon portion positioned close to the distal end, and (b) a marker portion having visual or tactile markings positioned proximal to the balloon portion.

2. The balloon catheter of claim 1 wherein the marker is an integral part of the catheter.

3. The balloon catheter of claim 1 wherein the marker is temporarily affixed to the exterior of the catheter.

4. The balloon catheter of claim 1 wherein the marker is a colored or raised band and is between about 2 to 3 cm proximal to the balloon portion.

5. The balloon catheter of claim 1 wherein the distal end of the catheter distal to the balloon portion is less than 1 cm in length.

6. The balloon catheter of claim 1 further comprising an inflation port.

7. The balloon catheter of claim 1 further comprising a pressure gauge that measures the pressure inside the balloon portion.

8. The balloon catheter of claim 1 further comprising a second lumen.

9. A kit comprising the balloon catheter of claim 1 and a stylet.

10. The kit of claim 7 wherein the stylet is plastic over a malleable metal core

11. The kit of claim 7 wherein the stylet comprises a distal end that is cushioned and devoid of metal.

12. The kit of claim 7 further comprising a traction collar for placement around the balloon catheter.
